(19) United States
(12) Patent Application Publication
(10) Pub. No.: US 2021/0290424 A1
(43) Pub. Date: Sep. 23, 2021

(54) AN ORTHOTIC PRODUCT

(71) Applicant: KINETIC ORTHOTICS PTY LTD, Marcoola (AU)

(72) Inventor: Dan Everson, Marcoola (AU)

(21) Appl. No.: 16/330,385

(22) PCT Filed: Sep. 6, 2017
(86) PCT No.: PCT/AU2017/000187

§ 371 Date: Mar. 5, 2019

(30) Foreign Application Priority Data
Sep. 6, 2016 (AU) 2016903571

(51) Int. Cl. A61F 5/14; A43B 5/12; A43B 7/14

(52) U.S. Cl. CPC A61F 5/14; A43B 7/1415; A43B 7/141; A43B 5/12

(57) ABSTRACT

An orthotic for a foot having a top side and a bottom side, the bottom side having flexion zone located between the medial and the lateral side of the orthotic at a position corresponding to the posterior metatarsal phalangeal joint of the foot and 25 mm behind; and/or the heel section of the bottom side of the orthotic pitched so that the height of the rear of the orthotic is greater than the innermost section of the heel; and/or the top side having a raised section located at a position corresponding to the medial side of the calcaneus of a foot.

### FIELD OF THE INVENTION

The present invention relates to orthotics.

### INCORPORATION BY REFERENCE

The present invention claims priority from Australian provisional application no 2016903571, the entire contents of which are hereby incorporated by reference.

### BACKGROUND OF THE INVENTION

Orthotics are used in the treatment of foot disorders. Such orthotics are often provided as an insert that can be placed within the patient's shoes.

Many existing orthotics consider only the contour of the foot and the position of the foot when sitting or standing.

It is only recently that developments in orthotics have begun to consider other aspects of the foot, such as the position and movement of the foot in locomotion. For example, the applicant's own Australian Patent Application No. 2012262646 describes a process in which an individual is assessed using a number of functional tests. The results of those tests can then be used to fit the individual with an orthotic that can further improve the efficiency of the individual's gait cycle and therefore mobility.

It is an object of this invention to provide an orthotic that has advantages over the prior art, or at least provides a useful alternative.

### SUMMARY OF THE INVENTION

This invention has come about due to the realisation of the inventor of the advantages of stimulating or blocking stimulation of certain parts of the foot, by means of targeted sections of an orthotic, to improve the movement pathway of an individual by improving the firing of the neuromuscular complex.

In a first aspect of the present invention, there is provided an orthotic having a top side and a bottom side, the orthotic further comprising one or more of the following features: (a) the bottom side having a flexion zone located from the medial to the lateral size of the orthotic at a position corresponding to the posterior metatarsal phalangeal joint of a foot and 25 mm behind; (b) the heel section of the bottom side of the orthotic pitched so that the height of the rear of the orthotic is greater than the innermost section of the heel; and (c) the top side containing a raised section located at a position corresponding to the medial side of the calcaneus of a foot.

In another aspect of the present invention, there is provided an orthotic having a top side and a bottom side, wherein the bottom side has an indentation located between the medial and the lateral side of the orthotic at a position corresponding to the posterior metatarsal phalangeal joint of the foot and 25 mm behind.

In another aspect of the present invention, there is provided an orthotic having a top side and a bottom side, wherein the heel section of the bottom side of the orthotic is pitched so that the height of the rear of the orthotic is greater than the innermost section of the heel.

In another aspect of the present invention, there is provided an orthotic having a top side and a bottom side, wherein the top side contains a raised section located at a position corresponding to the medial side of the calcaneus of a foot.

The raised section may be a substantially semicircular arc with a diameter of 2 to 4 cm.

The orthotic may further comprise a raised section in the top side of the orthotic located at a position corresponding to the cuboid area of the lateral side of the foot and extending anterior along one third of the shaft of the fifth metatarsal head, and a fascial groove located at a position corresponding to the area between the medial tubercle of the calcaneus to the insertion point of the medial tubercle into the base of the first metatarsal.

The orthotic may further comprise an indentation in the top side of the orthotic located at a position corresponding to the base of the first metatarsal of a foot.

The orthotic may further comprise a raised section located at a position corresponding to the sulci of the foot.

The orthotic may further comprise a raised section located at a position corresponding to 2 mm to 2 cm behind the second to fifth metatarsal heads of a foot.

For some applications, an orthotic of less than half length may be used, including a raised section located at a position corresponding to the medial side of the calcaneus of a foot.

Further aspects of the invention will become apparent from the embodiments described in the detailed description and drawings.

A detailed description of one or more embodiments of the invention is provided below, along with accompanying figures that illustrate by way of example the principles of the invention. While the invention is described in connection with such embodiments, it should be understood that the invention is not limited to any embodiment. On the contrary, the scope of the invention is limited only by the appended claims and the invention encompasses numerous alternatives, modifications and equivalents.

For the purpose of example, numerous specific details are set forth in the following description in order to provide a thorough understanding of the present invention. The present invention may be practiced according to the claims without some or all of these specific details. For the purposes of clarity, technical material that is known in the technical fields related to the invention has not been described in detail so that the present invention is not unnecessarily obscured.

### BRIEF DESCRIPTION OF DRAWINGS

Various embodiments/aspects of the invention will now be described with reference to the following drawings in which:

FIG. 1 is a bottom view of an orthotic in accordance with an embodiment of the present invention.

FIG. 2 is a top view of an orthotic in accordance with an embodiment of the present invention.

FIG. 3 is a diagram of the bones of a foot.

FIGS. 4 to 19 depict alternative embodiments of orthotics in accordance with the present invention.

### DETAILED DESCRIPTION

The present invention will now be described with reference to one or more embodiments of the invention.

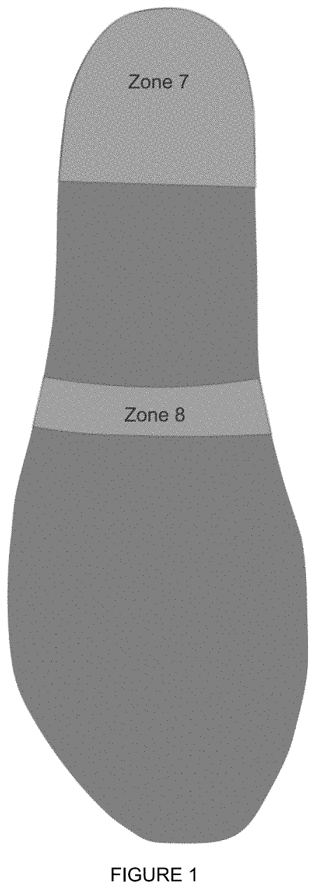

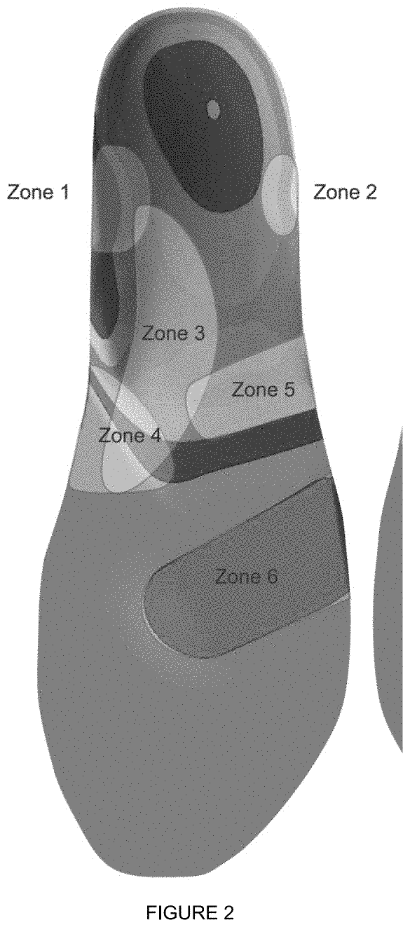

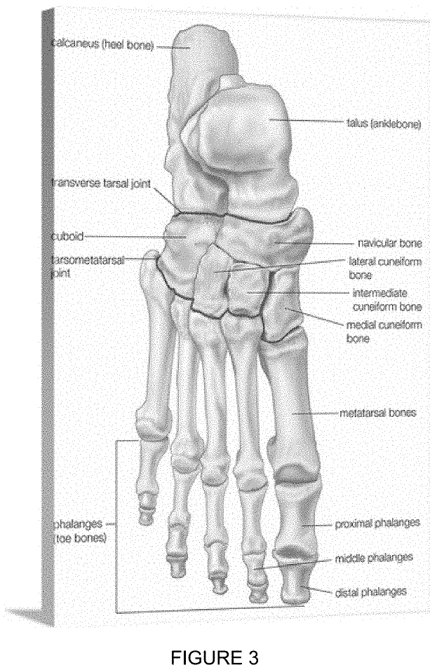

FIGS. 1 and 2 show the top side and bottom side of an orthotic in accordance with an embodiment of the present invention. The orthotic shows eight numbered zones, which correspond to particular features and locations on a patient's foot which have been identified as being important areas for neuro-stimulation. An orthotic in accordance with the present invention may have a raised section or groove, as appropriate, in one to eight of the identified zones.

The applicant has found that the stimulation or protection from stimulation, as appropriate, of particular locations of the foot corresponding to selected zone(s) improves the muscle function in order to achieve a more efficient gait cycle. Functional optimisation is achieved by improving the functional position of the foot. Firstly, by protecting and facilitating the windlass effect in conjunction with optimising the pivotal function at the key sagittal plane pivot sites in the foot; namely the heel, the ankle and the fore foot. Secondly, by stabilising the foot in the frontal plane in such a way as to create a functional equilibrium between the ability to efficiently pivot and the amount of support required (both in position and amplitude) to achieve this outcome.

A first zone is located in a position corresponding to the anterior of the medial side of the calcaneus and around the insertion site of the tibialis posterior into the side of the navicular of the foot. The zone is generally a substantially semicircular arc with a diameter that may vary from about 2 cm to about 4 cm. In an embodiment of the present invention there is provided an orthotic with a raised section in this zone, which increases the support of the orthotic to facilitate the tibialis posterior muscle group. It should be noted that, for some feet, the raised section in this zone may be slightly longer (longitudinally) than shown in the figures.

A second zone is located in a position corresponding to the area of the foot around the cuboid area of the lateral side of the foot and extending anterior along one third of the shaft of the fifth metatarsal head. In an embodiment of the present invention, a raised section is located in this zone. The stimulation of the foot by this raised zone works to stimulate the peroneal muscle insertions and tendons that track through this area. These insertions and tendons play an important role in stabilising the fore foot and the windlass effect during the propulsion phase of the gait cycle.

A third zone is a fascial groove zone starting in a location corresponding to the medial tubercle of the calcaneus to its insertion point into the base of the first metatarsal. The corresponding area on a foot is responsible for protecting the windlass effect. In an embodiment of the present invention, a groove is located in this zone. By having a groove in this zone, the pressure on the fascial chord of the foot can be minimised to reduce the incidence of uneven pressure on the chord interfering with the windlass mechanism.

A fourth functional zone is located at a position corresponding to the base of the first metatarsal zone and extending across approximately ⅓ of the width of the orthotic at the metatarsal area and one fifth of the length to the posterior heel point of the orthotic length on the medial side of the orthotic. In an embodiment of the invention, there is an indentation in the orthotic in this zone. This decrease in bulk behind the first metatarsal resulting from the indentation encourages the plantar flexion of the first metatarsal.

The fifth zone is positioned at a location corresponding to 2 mm to 2 cm behind the second to fifth metatarsal heads on a foot. In an embodiment of the present invention, there is a raised surface in this zone. This raised surface increases the tension on the digital muscles in the foot as they insert into the metatarsal heads.

The sixth zone is located at a position corresponding to the sulci of a foot. In an embodiment of the present invention, there is a raised surface in this zone. The raised surface engages with the foot to enable better digital traction and reduce clawing of the toes. The raised section in the sixth zone may provide a wedging effect—for example, it may be higher on the inside or outside of the foot, to improve the forefoot position. Increasing the height of the raised section on the inside of the foot can be used to invert the forefoot position, or vice versa. This function of wedging the ball of the foot is quite different to the use of wedges in ordinary orthotics, where the is positioned behind the metatarsal. Note that, although (in the figures) the zone is shown as only extending about halfway laterally across the orthotic, for some people (with more severe forefoot position issues) the raised surface may be extended all the way across the orthotic.

The seventh zone is located on the bottom of the orthotic of the present invention in a position corresponding to the plantar heel surface of the foot. In an embodiment of the present invention, this zone is pitched from the rear of the orthotic to the front of the heel section. The pitched heel raise facilitates angle joint dorsiflexion.

The eighth zone is located on the bottom of the orthotic in a position corresponding to the location between the metatarsal phalangeal joints of the foot to 25 mm behind the phalangeal joints, and extends from the medial to the lateral size of the orthotic. In an embodiment of the present invention, an indentation extends across this entire zone. The presence of this indentation in the orthotic improves the rocker function in the forefoot to improve the efficiency of the gait of an individual and reduce the soft tissue stress on the foot. Although the feature has been described above as an indentation, in other embodiments it may simply be a flexion zone or hinge point, defined by a weakness or other arrangement (for example use of a more resilient material in the orthotic).

For each raised surface, the height of the raise is preferably greatest towards the centre of the respective zone—that is, there is a peak towards the centre of the zone. Each peak may be raised between 1 mm and 25 mm from the surrounding orthotic surface, but is preferably between 2 mm and 20 mm. and in most circumstances will be preferably between 3 mm and 8 mm.

Orthotics may be pre-made with the raised surfaces or grooves in all or most of the above-designed zones as it is anticipated that almost all patients in need of orthotics would benefit from stimulation/blocking of stimulation (as appropriate) in the identified zones.

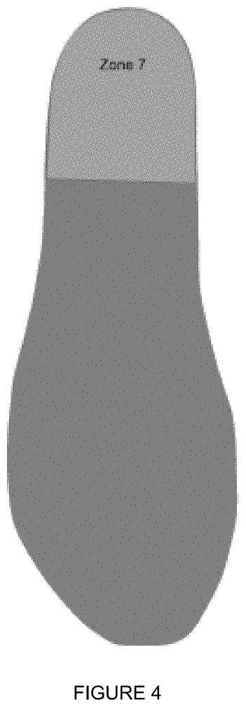

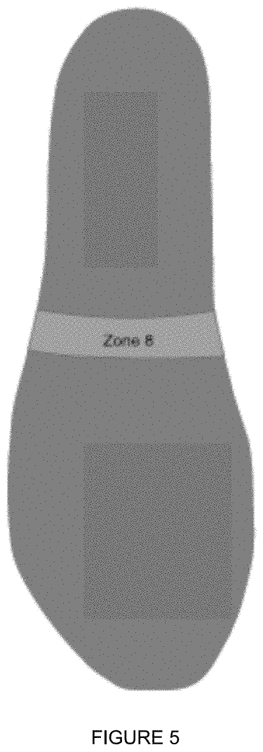

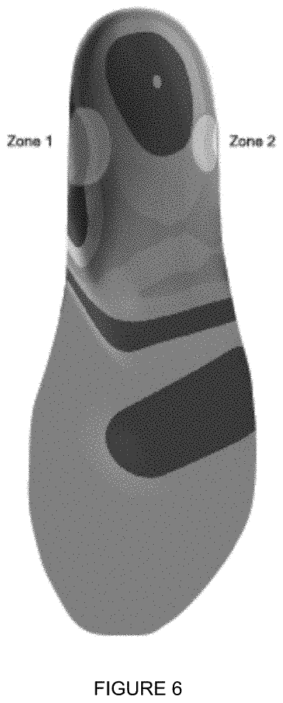

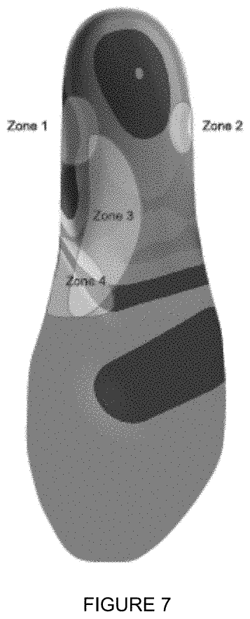

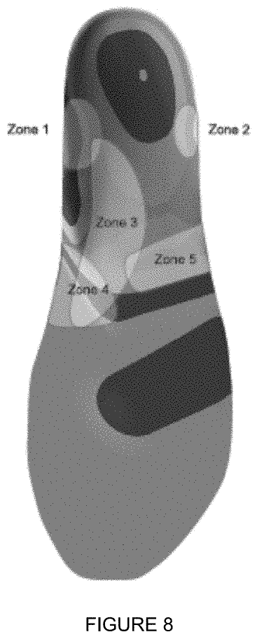

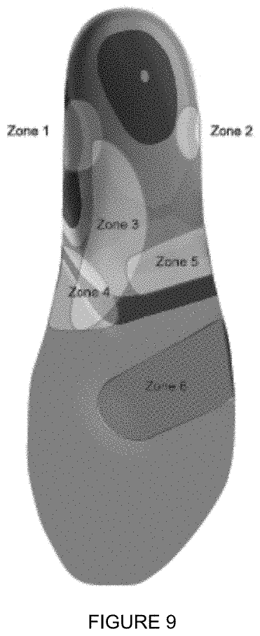

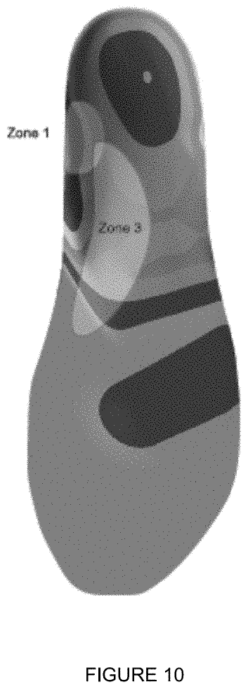

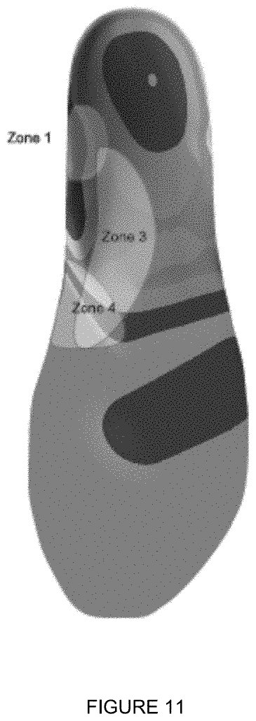

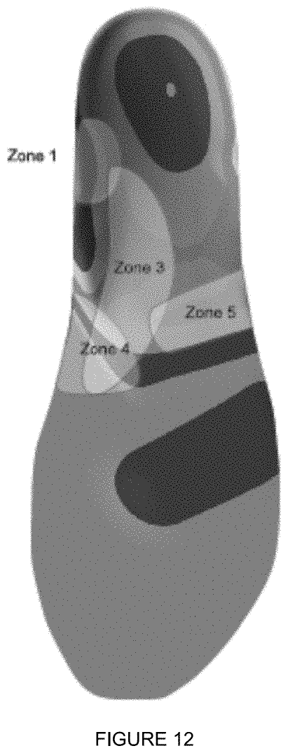

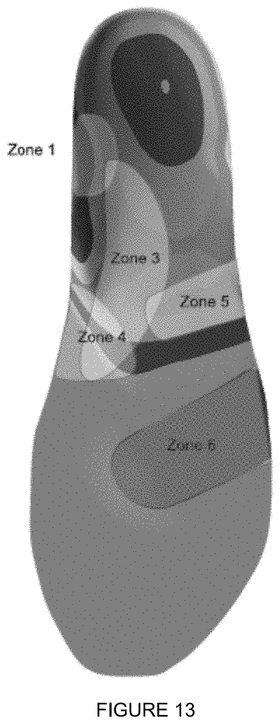

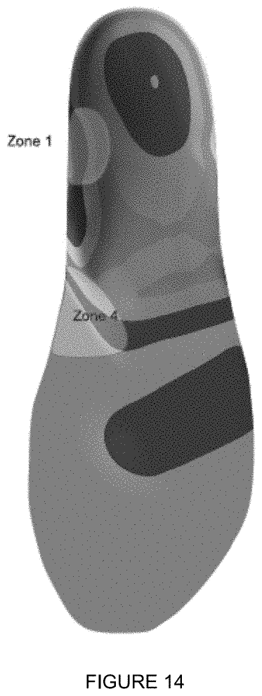

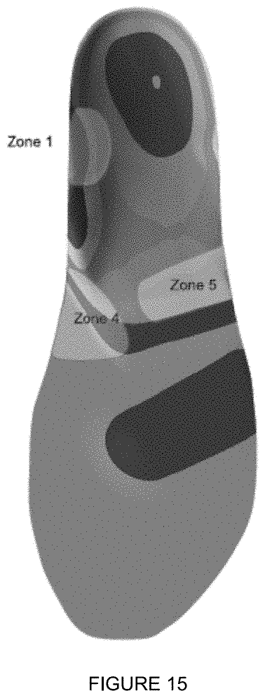

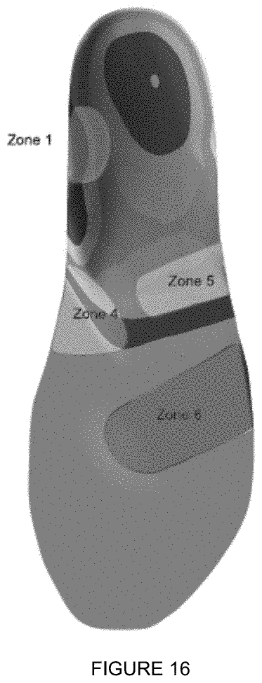

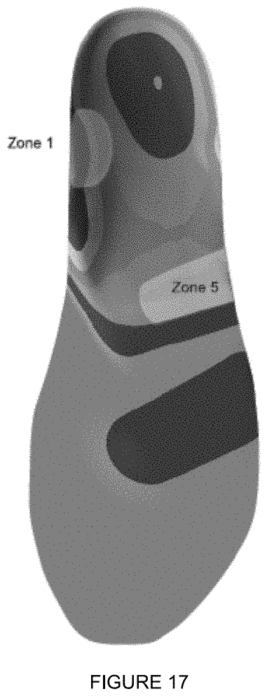

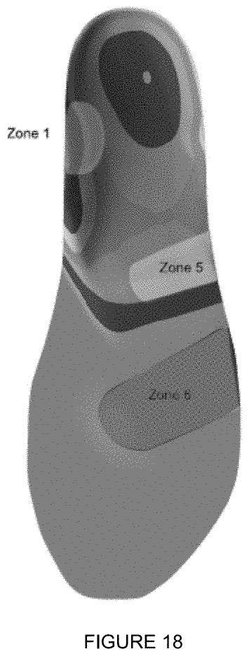

Alternatively, custom orthotics may be manufactured which include, amongst any other features suggested by the customised analysis, raised surfaces or grooves (as appropriate) in all or some of the above identified zones. For example, a patient may have a need for stimulation/blocking (as appropriate) in zones 7 only (FIG. 4), zone 8 only (FIG. 5), zones 1 and 2 only (FIG. 6), zones 1 to 4 (FIG. 7), zones 1 to 5 (FIG. 8), zones 1 to 6 (FIG. 9) zones 1 and 3 only (FIG. 10), zones 1, 3 and 4 (FIG. 11), zones 1, 3, 4 and 5 (FIG. 12), zones 1, 3, 4, 5 and 6 (FIG. 13), zones 1 and 4 only (FIG. 14), zones 1, 4 and 5 (FIG. 15), zones 1, 4, 5 and 6 (FIG. 16), zones 1 and 5 only (FIG. 17) or zones 1, 5 and 6 (FIG. 18).

The orthotics shown in FIGS. 4 to 18 are all full length orthotics. However, shorter orthotics (e.g. ¾ length, half-length, even ¼ length orthotics) may also be used in different embodiments of the invention, although shorter orthotics may not include all of the zones toward the front of the orthotic.

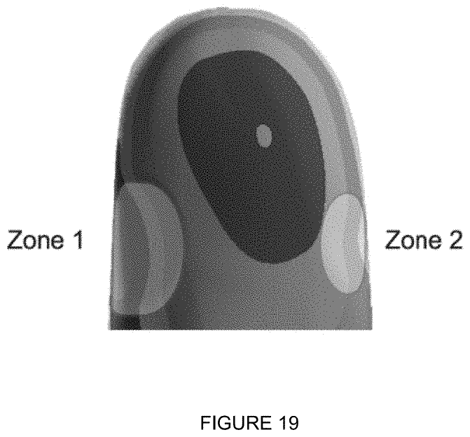

In FIG. 19, there is depicted an orthotic having particular application in ballet, and other environments where small footwear worn such as in sprinting, cycling, or fashion. The orthotic in this embodiment is less than half length, and so takes up little room within the shoes and only has the first (calcaneal) and second (cuboid) “bumps” (ie raised sections). At least for a ballet application, no or minimal pitched heel may be used on the bottom of the orthotic. However, by stimulating of the foot using these bumps in zones 1 and 2, improved performance may be achieved in these applications.

The word ‘comprising’, and forms of the word ‘comprising’, when used in this specification is taken to specify the presence of stated features, integers, steps or components but does not preclude the presence or addition of one or more other features, integers, steps, components or groups thereof.

In this specification where a document, act or item of knowledge is referred to or discussed, this reference or discussion is not an admission that the document, act or item of knowledge or any combination thereof was at the priority date, publicly available, known to the public, part of the common general knowledge; or known to be relevant to an attempt to solve any problem with which this specification is concerned.

1. An orthotic for a foot having a top side and a bottom side, the orthotic further comprising one or more of the following features:
(a) the bottom side having flexion zone located between a medial and a lateral side of the orthotic at a position corresponding to a posterior metatarsal phalangeal joint of the foot and 25 mm behind;
(b) a heel section of the bottom side of the orthotic pitched so that a height of a rear of the orthotic is greater than an innermost section of the heel; or
(c) the top side having a raised section located at a position corresponding to a medial side of a calcaneus of a foot.

2. The orthotic of claim 1, wherein the raised section is a substantially semicircular arc with a diameter of 2 to 4 cm.

3. The orthotic of claim 1, further comprising a raised section in the top side of the orthotic located at a position corresponding to a cuboid area of a lateral side of the foot and extending anterior along one third of a shaft of a fifth metatarsal head, and a fascial groove located at a position corresponding to an area between a medial tubercle of the calcaneus to an insertion point of a medial tubercle into a base of a first metatarsal.

4. The orthotic of claim 3, further comprising an indentation in the top side of the orthotic located at a position corresponding to the base of the first metatarsal of a foot.

5. The orthotic of claim 1 further comprising a raised section located at a position corresponding to a sulci of the foot.

6. The orthotic of claim 1 further comprising a raised section located at a position corresponding to 2 mm to 2 cm behind the second to fifth metatarsal heads of the foot.

7. The orthotic of claim 1, wherein a height of the raised section is greatest towards the center of the flexion zone.

8. The orthotic of claim 1, wherein the height of the raised section is less than 25 mm.

9. The orthotic of claim 1, wherein the height of the raised section is between 2 mm and 20 mm.

10. The orthotic of claim 1, wherein the height of the raised section is between 3 mm and 8 mm.

11. An orthotic for a foot for ballet, the orthotic being less than half a length of the foot, a top side of the orthotic including a raised section located at a position corresponding to a medial side of a calcaneus of the foot.

12. The orthotic of claim 11, wherein the raised section is a substantially semicircular arc with a diameter of 2 to 4 cm.

13. The orthotic of claim 11, further comprising a raised section in the top side of the orthotic located at a position corresponding to a cuboid area of a lateral side of the foot and extending anterior along one third of a shaft of a fifth metatarsal head.
